(19) United States
(12) Patent Application Publication
(10) Pub. No.: US 2026/0215928 A1
(43) Pub. Date: Jul. 30, 2026

(54) SPINAL REPAIR DEVICE

(71) Applicant: OrthoSon Limited, Oxford (GB)

(72) Inventors: Nick Birch, Elkington (GB); Ross Laska, Oxford (GB); Olga Boubriak, Oxford (GB); Martin Gardener, Oxford (GB)

(73) Assignee: OrthoSon Limited, Oxford (GB)

(21) Appl. No.: 19/148,977

(22) PCT Filed: Jan. 17, 2024
(86) PCT No.: PCT/GB2024/050122

§ 371 Date: Jul. 17, 2025

(30) Foreign Application Priority Data
Jan. 18, 2023 (GB) 2300746.1

(51) Int. Cl. A61F 2/44; A61F 2/30; A61L 27/44; A61L 27/52; A61L 27/54

(52) U.S. Cl. CPC A61F 2/442; A61L 27/44; A61L 27/52; A61L 27/54; A61F 2002/30299; A61F 2002/30308; A61F 2002/30677; A61L 2430/02

(57) ABSTRACT

The present invention provides a spinal repair device, a method of preparing the same, and a method of treating a subject using the same.

### FIELD OF THE INVENTION

The present invention relates to a spinal repair device, a method of preparing the spinal repair device, and a method of treating a subject using the spinal repair device.

### BACKGROUND TO THE INVENTION

It is estimated that at least 500 million people suffer with low back pain globally. Low back pain is often caused by the degeneration of one or more intervertebral discs. Despite the large number of patients and the significant impact on quality of life, when conservative therapy such as self-management, physiotherapy and chiropractic is unsuccessful, other options can be limited, and are often invasive, high-cost procedures associated with significant complications and having poor correlation with diagnosis.

In recent years, technological developments have been aimed at treating degenerated intervertebral discs in a less invasive and more effective manner, for instance via percutaneous nucleus pulposus replacement—a procedure that removes and replaces a degenerated nucleus pulposus. However, these procedures leave small punctures in the annulus fibrosus which need to be sealed.

Additionally, it is possible for annular defects to arise de novo, for instance, during strenuous activity. Annular defects are defects in the annulus fibrosus, and may be tears, fissures, holes, and the like.

Annular defects cause symptoms such as back pain and leg pain, and can also lead to intervertebral disc degeneration.

Various devices for repairing annular defects are known in the art. For instance, angle-ply patches made from animal pericardium or biological adhesive are known. Other devices consist of a patch applied to the outside of the annulus over the defect by either suturing in place or by adhesion. Other devices act as plugs within an annular defect that are held in place by adhesive. However, known devices are associated with various shortcomings, in particular the risk of device expulsion. Therefore additional measures are typically needed to reduce this risk. Known devices therefore may involve invasive implantation procedures, such as the use of sutures, tension bands, staples, or any other type of fixation means known in the art to secure the device in place and/or close the annular defect. This may lead to a greater risk of harm to the patient, low efficacy, and/or high cost.

Therefore, there remains a need to provide a safer, cost-effective treatment to restore spine function and alleviate symptoms for patients living with persistent, discogenic low back pain.

### SUMMARY OF THE INVENTION

To solve this problem, the present inventors have devised a new spinal repair device that can be used to repair annular defects in a safe, efficacious and cost-effective manner which reduces the requirement for invasive procedures. The present invention therefore provides a safer, cost-effective treatment to restore spine function and alleviate symptoms for patients living with persistent, discogenic low back pain.

One of the main issues that intervertebral implants face is poor efficacy due to expulsion during use, for instance when forces impact on the implant when bending, twisting, and so on. To solve this problem the present inventors have devised a new spinal repair device having a specific design and shape, and have surprisingly found that this specific design and shape addresses the challenging hurdle of implant expulsion. In particular, when pressure is applied to an intervertebral disc comprising an implant, pressure is similarly applied to the implant itself. For many known implants, this pressure either loosens or completely removes the implant from the desired location, thus reducing or nullifying its efficacy. In the case of the present invention, however, that pressure is transferred away from the implant via the head of the device and on to the annulus fibrosus. This pressure itself therefore acts to lock the device in place.

The design and shape of the device mean that the pushing of the device against the surrounds of the annular defect provides effective and robust expulsion and herniation resistance, especially in the medium-term.

Preferably, the short-term expulsion resistance of the device is further enhanced. This is achieved when the device comprises a sticky material, in particular one adhesive to collagen, which is preferred.

Preferably, the long-term expulsion resistance of the device is also further enhanced. This may be achieved when the device comprises a material that is susceptible to cell integration and/or compatible with the extracellular matrix, which material may be the same as or different to the sticky material. For example, when the material of the device is a benign environment for the in-growth of new annulus fibrosus and/or nucleus pulposus cells, then the growth of these new cells within the device further increases integration with the surrounding tissues and so further reduces the risk of expulsion, as well as making the device an integral part of the restored annulus. In a further aspect, the device may comprise one or more agents that positively encourage cell growth and/or integration with the surrounding tissues.

The device of the present invention is highly resistant to expulsion from the intervertebral disc. Furthermore, unlike all externally applied patches, such as those commonly used in the field at present, the design of the device of the present invention means that the device actually becomes more likely to resist herniation of nuclear contents with increasing load/pressure.

Another advantage of the present invention is that there is no requirement for sutures, surgical screws, tension bands, staples, or any other type of fixation means known in the art to secure the device in place. The device of the present invention is typically wholly free of fixation means such as sutures, surgical screws, tension bands, staples, and so on. Compared to known spinal repair devices, insertion of the device of the present invention may therefore significantly less invasive, and inherently safer for the patient. Alternatively, the device of the invention may include one or more fixation means to further improve its resistance to expulsion from the intervertebral disc.

A further advantage of the present invention is that there is no requirement for separate adhesive to be used with the device of the present invention. That is, there is no requirement for separate adhesive to hold the device of the invention in place. Typically, therefore, the device of the present invention does not use any separate adhesive.

When in use, the device of the present invention reduces the risk of, or completely prevents, the herniation of nucleus pulposus material, whether native or implanted, through defects in the annulus fibrosus of the intervertebral disc. As such, the device of the present invention repairs annular defects, and so can be used to restore an annulus.

The device of the present invention can be applied to the closure of annular defects caused by procedures to treat herniation of the nucleus pulposus, such as microdiscectomy. It can also be applied to annular defects that arise de novo and cause symptoms such as back pain and leg pain. It can also be applied as part of a whole disc restoration procedure, such as when a painful degenerate disc is treated with an implant to the nucleus pulposus through a portal in the annulus fibrosus, and the annulus portal needs to be sealed at the end of the procedure.

The present invention provides the following aspects:
[1]A spinal repair device for repair of a defect in an annulus fibrosus of an intervertebral disc, the intervertebral disc having an annulus fibrosus and a nucleus pulposus, the device including at least:
   (a) a head having a surface, and
   (b) a body having a major axis;
   wherein the body is connected to the surface;
   wherein the body is arranged to be positioned within the defect, and the head is arranged to be positioned within the nucleus pulposus such that its surface is adjacent to the surface of the annulus fibrosus and such that the head extends beyond the periphery of the defect; and
   wherein the head comprises a deformable nanocomposite hydrogel, the hydrogel comprising polymers of one or more water soluble monomers.
[2]A spinal repair device including at least:
   (a) a head comprising a surface, and
   (b) a body having a major axis;
   wherein the body is connected to the surface and configured such that its major axis is approximately perpendicular to the surface, the major axis defining a plane perpendicular thereto, the projection of the head on the plane extending beyond the projection of the body on the plane; and
   wherein the head comprises a deformable nanocomposite hydrogel, the hydrogel comprising polymers of one or more water soluble monomers.
[3] The spinal repair device of [1] or [2], wherein the body comprises a deformable nanocomposite hydrogel, the hydrogel comprising polymers of one or more water soluble monomers.
[4] The spinal repair device of any preceding aspect, wherein the hydrogel further comprises one or more crosslinking agents.
[5] The spinal repair device of [4], wherein the one or more crosslinking agents are selected from the following group or any combination thereof: clay particles, aldehydes, ketones, carboxylic acids, amines, amides, esters, alkali metal salts and alkali earth metal salts, preferably wherein the one or more crosslinking agents comprise clay particles.
[6] The spinal repair device of [4] or [5], wherein the clay particles comprise particles of one or more of the following clays: montmorillonite, hectorite, saponite, vermiculite, mica, bentonite, and a fibrous clay, preferably wherein the clay particles comprise particles of hectorite.
[7] The spinal repair device of any preceding aspect, wherein the hydrogel polymers are polymers of one or more water soluble monomers selected from the following group acrylic acids, acid anhydrides, acrylates, sulfonic acids, vinyl sulfonates, pyrrolidones, N-isopropylacrylamide (NIPAM), N,N-dimethylacrylamide (DMAC), glycerol monomethacrylate, hydroxyl ethyl methacrylate, polyethyleneglycomethacrylate, vinyl pyrrolidone, vinyl acetate, ethylene glycol, and styrene sulphonic acid, preferably wherein the one or more water soluble monomers comprise NIPAM, DMAC, vinyl acetate, ethylene glycol, and/or N-vinyl pyrrolidone, more preferably wherein the one or more water soluble monomers comprise NIPAM and optionally also DMAC.
[8] The spinal repair device of any preceding aspect, wherein the polymer comprises a first and a second water soluble monomer, wherein the first and second water soluble monomers are present in a mass ratio of from 100:1 to 3:1, preferably from 50:1 to 5:1, and wherein the first monomer is preferably NIPAM, and the second monomer is preferably DMAC.
[9] The spinal repair device of any preceding aspect, wherein the deformable nanocomposite hydrogel further comprises one or more of the following additional agents: mammalian cells such as stem cells, an antimicrobial, an antibody, a bacteria, a protein, a pharmaceutical, a peptide, another water soluble polymer, hyaluronic acid, hydrophilic gels, polyethylene glycol, polypropylene glycol, a mixture of polyethylene glycol and polypropylene glycol, cellulose ethers, chitosan, alginates, proteoglycans, natural starches, collagen, gelatine, hydroxyapatite, a thickener, a rheology modifier, a surfactant, a pigment, a dye, and a radio opaque material.
[10] The spinal repair device of any preceding aspect, wherein the head is hemispherical in shape and has a curved surface and a flat surface, the body being connected to the flat surface.
[11] The spinal repair device of any preceding aspect, wherein the head is deformable to a size that is from about a third to about a fifth of its original size by volume.
[12]A spinal repair device obtainable by dehydrating the spinal repair device of any preceding aspect.
[13]. A method of producing a spinal repair device according to any one of [1] to [12] comprising the following steps:
   preparing an aqueous suspension containing (i) dispersed clay particles, (ii) one or more water soluble monomers, and (iii) one or more radical initiators capable of free radical disassociation to form a composite hydrogel precursor liquid;
   dissociating the one or more radical initiators to provide free radical polymerisation of the one or more monomers at a temperature above a gelation temperature of a resulting aqueous polymer-clay precursor hydrogel;
   optionally maintaining the polymer-clay precursor hydrogel in a fluid phase above the gelation temperature;
   processing the polymer-clay precursor hydrogel into the desired shape; and
   reducing the temperature of the polymer-clay precursor hydrogel below its gelation temperature;
   wherein the precursor is processed prior to and/or during the step of reducing the temperature thereof.
[14] The method of [13], wherein the one or more radical initiators are selected from the following group azobisisobutyronitrile (AIBN), 2,2′-azobis(2-methylpropionamide) (AIBA) and cyanovaleric acid (CVA), preferably wherein the one or more radical initiators comprise AIBN.
[15] The method of [13] or [14], wherein the polymer-clay precursor hydrogel is processed by cast moulding, spinning, foaming, coating, 3D printing, or spraying, preferably by cast moulding.
[16]A method of repairing an annular defect in a subject, the method comprising inserting the device of any one of [1] to [12] into the annular defect such that the body of the device is situated partially or completely in the annular defect and the head is situated inside the annulus fibrosus.
[17] The method of [16], wherein the insertion comprises
   reducing the size of the head of the device of any one of [1] to [12]; and
   inserting the head through the annular defect;
   wherein the size of the head is reduced prior to and/or during the insertion.
[18] The method of [17], wherein the size of the head is reduced such that it can be inserted through the defect, preferably wherein the size of the head is reduced to from about a third to about a fifth of its original size.
[19] The method of any one of [16] to [18], wherein the insertion does not worsen the defect.
[20] The method of any one of [17] to [19], wherein the size of the head is reduced by deforming the nanocomposite hydrogel.
[21] The method of any one of [17] to [19], wherein the size of the head is reduced by dehydrating the nanocomposite hydrogel prior to insertion.
[22] The method of any one of [16] to [21], wherein the treatment is part of a whole disc restoration procedure that also involves treating a degenerate disc with an implant to the nucleus pulposus.
[23] The method of [22], wherein the implant is implanted either before, concurrently or after the device is inserted into the annular defect.
[24] The method of [22] or [23], wherein the device binds chemically to the implant.
[25] The method of any one of [16] to [24], wherein the condition to be treated is one or more of the following: back pain in particular lower back pain, neck pain, leg pain, reduced spine function, reduced mobility.

### BRIEF DESCRIPTION OF THE FIGURES

FIG. 1 shows an embodiment wherein the device has a mushroom shape. FIG. 1A is a perspective view of the device, FIG. 1B is a front view of the device, FIG. 1C is a plan view of the plane, and FIG. 1D is another front view of the device.

FIG. 2 shows an embodiment wherein the device has a T-bar shape. FIG. 2A is a perspective view of the device, FIG. 2B is a front view of the device, FIG. 2C is a plan view of the plane, and FIG. 2D is another front view of the device.

FIG. 3 shows an embodiment wherein the device has an anchor shape. FIG. 3A is a perspective view of the device, FIG. 3B is a front view of the device, FIG. 3C is a plan view of the plane, and FIG. 3D is another front view of the device.

FIG. 4 is a cross-section of the device in the xy-plane, and depicts the deformation of the device under pressure. The fact that the device has a mushroom shape in this figure is merely illustrative.

FIG. 5 depicts three different head and body projections on the plane perpendicular to the major axis. In FIG. 5A, the projection of the head extends from a position corresponding to the centre of the body twice as far as the body. In FIG. 5B, the projection of the head extends from a position corresponding to the centre of the body three times as far as the body. In FIG. 5C, the projection of the head extends further in the x-direction of the plane than it does in the z-direction.

### DETAILED DESCRIPTION

### Definitions

Unless otherwise indicated, the terms used within this document shall have their ordinary meaning as understood by the person skilled in the art. However, as used in the present invention, the particular terms given in the non-exhaustive below shall have the following definitions.

Combination: any possible selection of options from a given group. This includes multiple different options. For instance, the wording “one or more agents selected from the following group or any combination thereof: A, B, C” encompasses the following selections: A, B, C, A+B, A+C, B+C, and A+B+C.

Deform: decrease the size and/or alter the shape of. This can be achieved by any means, including mechanical pressure, chemical reaction, and physical reaction, as well as any combination thereof. Typically deformation is achieved by mechanical pressure.

Deformable: readily deformed without the exertion of substantial force. A deformable material may have a Young's Modulus of from 0.01 MPa to 2 GPa, preferably from 0.1 MPa to 0.1 GPa, more preferably from 0.5 MPa to 100 MPa, such as from 1 MPa to 10 MPa. Typically, this corresponds to the “deformable” material being less stiff than the material of the annulus fibrosus, such that it is the former that deforms during insertion of the head of the device through the annular defect, rather than the annulus tissues being pushed out of the way by the head of the device.

Annulus fibrosus: the outer fibrous ring of the intervertebral disc. The terms “annulus” and “annulus fibrosus” may be used synonymously and interchangeably throughout the specification.

Nucleus pulposus: the inner gel-like centre of the intervertebral disc. The terms “nucleus” and “nucleus pulposus” may be used synonymously and interchangeably throughout the specification.

Annular defect: any defect passing through the annulus fibrosus. This includes tears, rips, fissures, holes, punctures, and so on. The defect may be small, large or medium-sized.

Dehydrated material: a material that has had some or all of its water removed. After the water has been removed, a dehydrated material may have a total mass that is equivalent to 30 wt. %, 50 wt. %, 70 wt. %, or 90 wt. % of the hydrated material. The extent of dehydration can readily be measured by the skilled person in any routine manner, for instance by weighing the material before and after dehydration to determine the total mass reduction, or using Karl-Fischer analysis.

Rehydrated material: any dehydrated material that has taken in water. The amount of water taken in may be less than, equal to, or greater than the amount of water initial removed during dehydration. After the water has been taken up, the rehydrated material may have a total mass that is equivalent to 200 wt. %, 500 wt. %, 1000 wt. %, or 2000 wt. % of the dehydrated material. The extent of rehydration can readily be measured by the skilled person in any routine manner, for instance by weighing the material prior to and after rehydration to determine the total mass gain.

Hydrogel: a crosslinked hydrophilic polymer that does not dissolve in water. Unless otherwise indicated, the hydrogels discussed herein are hydrated, i.e. they are not dehydrated.

Dehydrated hydrogel: A hydrogel that has been dehydrated per the definition of a dehydrated material above.

Polymer of one or more monomers: polymer obtained from, or obtainable by, the polymerisation of said one or more monomers.

Swelling: increasing in size, for instance due to water absorption. For example, the head of the device will often swell under physiological conditions to improve the ability of the head to block the annular defect.

Repair: in the context of treating annular defects, “repair”/“repairing” and the like may be used to mean completely fix and/or restore function to the intervertebral disc by effectively nullifying the annular defect. Alternatively, it may mean significantly or substantially reducing the amount of nuclear material that can escape through the annular defect to a manageable level. Other words such as “closing”, “fixing”, “blocking”, and the like may be used synonymously.

Sticky: adhesive, especially to collagen, such that the resistance of the device to expulsion from the intervertebral disc is increased, particularly in the short term.

Herniation: as used herein this refers to nuclear material pushing through an annular defect.

Expulsion: repair devices/implants being dislocated. This includes devices being pushed out of position, as well as devices being expelled from the intervertebral disc entirely.

### Spinal Repair Device

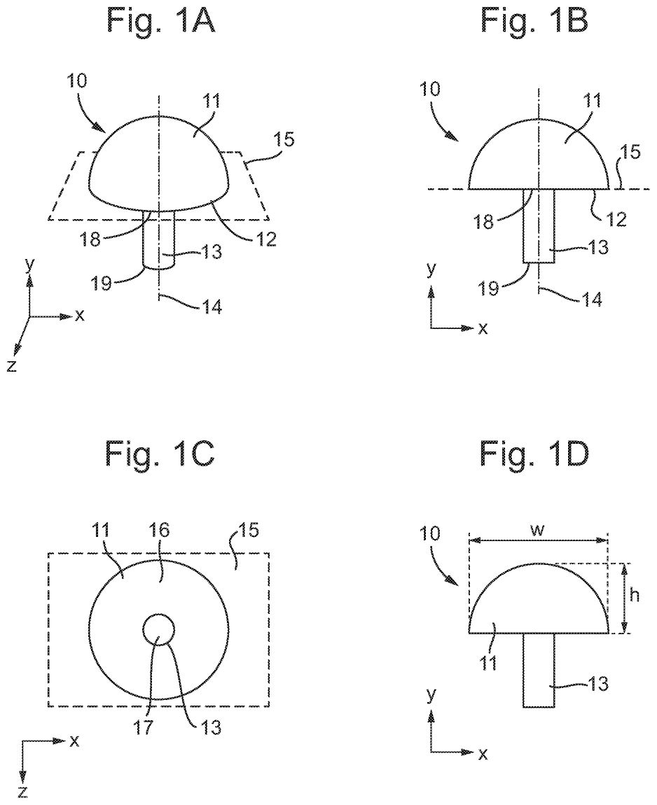

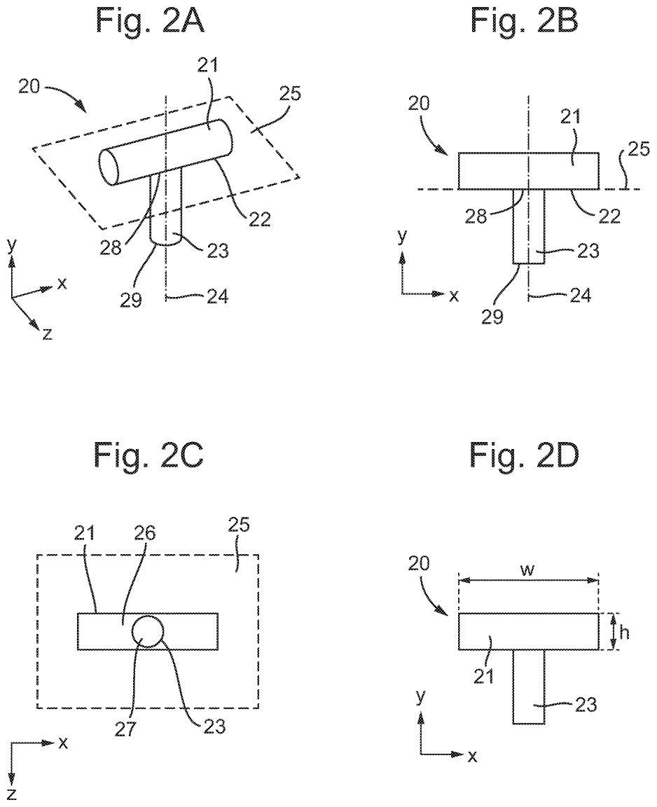

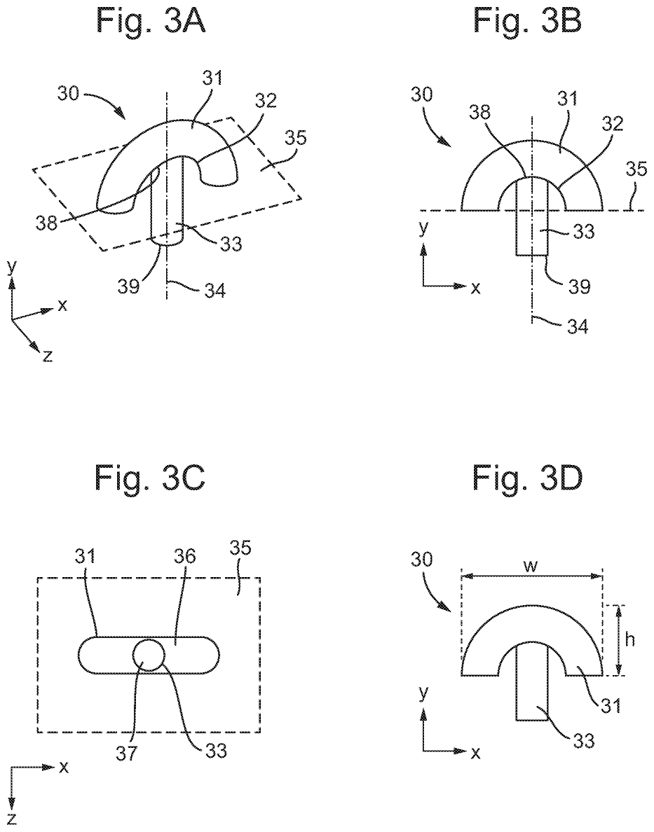

With reference to FIGS. 1 to 3, the present invention provides a spinal repair device 10, 20, 30 including at least a head 11, 21, 31 and a body 13, 23, 33 having a major axis 14, 24, 34, wherein the body is connected to the head. The head has a surface 12, 22, 32 and the body is typically joined to the head at the surface. The body is typically configured such that its major axis is approximately perpendicular to the surface. The major axis defines a plane 15, 25, 35 that is perpendicular to the major axis, the projection of the head 16, 26, 36 on the plane extending beyond the projection of the body 17, 27, 37 on the plane. In the Figures, the major axis is aligned with the y-axis and the perpendicular plane corresponds to the xz-plane.

The body 13, 23, 33 is arranged to be positioned within an annular defect. The head 11, 21, 31 is arranged to be positioned within the nucleus such that its surface 12, 22, 32 is adjacent the surface of the annulus. The projection of the head on the plane perpendicular to the major axis of the body ensures that the head extends beyond the periphery of the annular defect. Therefore, the invention also provides a spinal repair device including at least (a) a head having a surface, and (b) a body having a major axis, wherein the body is connected to the surface, wherein the body is arranged to be positioned within an annular defect, and the head is arranged to be positioned within the nucleus (i.e. within the interior of the intervertebral disc) such that its surface is adjacent to the surface of the annulus and such that the head extends beyond the periphery of the annular defect (i.e. approximately in the direction of the centre of the intervertebral disc), wherein the head comprises a deformable nanocomposite hydrogel, the hydrogel comprising polymers of one or more water soluble monomers. Typically, the head is arranged such that is may be deformed sufficiently to pass through the annular defect and re-expand once it has passed through the defect.

The body has a proximal end 18, 28, 38 and a distal end 19, 29, 39. The proximal end is connected to the head, and the distal end is distal thereto. The position of the plane is fixed insofar as the plane is perpendicular to the major axis of the body. However, the plane may be located at the proximal end of the body, at the distal end of the body, or at another location.

As shown in FIGS. 1D, 2D, 3D and 4, the head has a height h and a width w. The width w is measured at the widest point of the head. When viewing the device in the xy-plane as shown in FIG. 1—wherein the major axis of the body aligns with the y-axis and typically the greatest extension of the projection of the head beyond the projection of the body on the plane aligns with the x-axis (e.g. as shown in the Figures)—the height h is the distance along the y-axis between the surface and the highest point of the head, and the width w is the distance along the x-axis between the leftmost part of the head and the rightmost part of the head.

The body may be connected to the surface of the head at approximately the centre of the head, preferably at the centre of the head, such that the device is typically approximately symmetrical about the major axis of the body when viewed in the xy-plane as defined above (i.e. as shown in the Figures). Alternatively, the body may be connected to the surface of the head away from the centre of the head. For instance, the body may be connected to the surface of the head at a distance of 0.1 w, 0.2 w, or 0.3 w away from the centre of the head. Viewing the device in the xy-plane as defined above, this displacement may be along the x-axis, but equally may also be in a different direction.

The angle between the surface of the head and the major axis of the body is typically perpendicular, but may deviate from exactly 90° (i.e. the perpendicular). For instance, the angle may deviate from 90° by up to 15°, up to 30°, or up to 45°. The angle between the major axis of the body and the surface being approximately perpendicular therefore includes angles of from 45° to 135°.

The surface may be flat or approximately flat, or may be curved. Where the surface is curved, approximately perpendicular indicates that the major axis is approximately normal to the surface. Hence, where the surface is curved, approximately perpendicular may indicate that the major axis is aligned approximately radially with the surface, as opposed to being aligned obliquely with the surface.

Typically, the body and head are pre-formed as opposed to made in situ. The body and head are also typically comprised within a single component. For example, the body and head may be made together in a single step and/or be made from the same material.

The invention surprisingly solves the problem of reducing or fully preventing herniation of nucleus material, whether native or implanted, from the intervertebral disc through defects in the annulus fibrosus by utilising a device that is inserted into the defect. As such, the presence of the device plugs the defect in the annulus reducing the risk of further herniation of the nucleus.

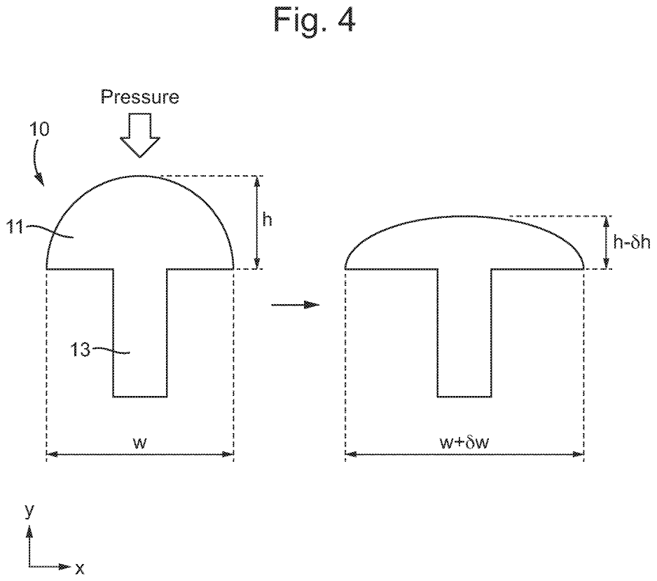

When a force is applied on the intervertebral disc, for instance when the subject is twisting their spine, the nucleus pulposus acts as a shock absorber. However, the presence of an annular defect provides a weak point in the annulus fibrosus, and the nucleus pulposus pushes against this weak point, and nuclear material may escape the disc via the defect. Additionally, if an externally applied device is used to patch the annular defect, there is a risk of the externally applied device being dislodged or completely expelled from its position, in particular when under load, and so failing to properly repair the annular defect. By contrast, the device of the present invention is positioned within the annulus in use, such that the head of the device is in contact with the nucleus pulposus, and the body of the device is located partially or completely in the annular defect. When the nucleus pulposus applies pressure to the head of the device, the head of the device deforms, compressing in a direction parallel to the axis of the body, as depicted by the arrow in FIG. 4. The head of the device has a greater projection than the body perpendicular to the axis of the body, such that the pressure created by this compression is applied on to the surrounds of the annular defect, and serves to lock the device in place. With reference to FIG. 4, the compression may cause the height of the head of the device h to decrease slightly by δh, and the width of the head w to increase slightly by δw, thus spreading the pressure across the surrounds of the annular defect.

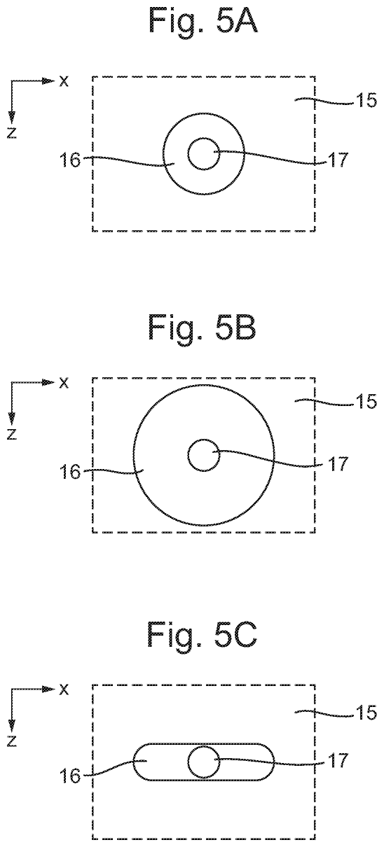

The shape of the head of the device is not particularly limited, so long as the projection of the head on the plane perpendicular to the major axis extends beyond the projection of the body on the plane. The degree that the projection of the head on the plane perpendicular to the major axis extends beyond the projection of the body on the plane is not particularly limited, and may be tailored to suit the specific size and shape of the annular defect to be treated. For instance, from a position corresponding to the centre of the body, the projection of the head on the plane typically extends by 2 to 10 times as far as the projection of the body on the plane. Preferably, the projection of the head 16 on the plane 15 perpendicular to the major axis extends from a position corresponding to the centre of the body at least twice as far as the projection of the body 17 (see FIG. 5A), more preferably at least three times as far as the projection of the body (see FIG. 5B), such as at least five times as far.

Advantageously, the projection of the head on the plane perpendicular to the major axis extends beyond the projection of the body on the plane in two perpendicular dimensions in said plane, i.e. in both the x and z dimensions as shown in FIGS. 5A and 5B. However, it is also possible for the projection of the head to extend beyond the projection of the body on the plane in only one perpendicular dimension in said plane. This is illustrated in FIG. 5C. Preferably, the projection of the head on the plane perpendicular to the major axis extends beyond the projection of the body on the plane in all dimensions in said plane. Thus, for example, the projection of the head may form a cross, or preferably a circle, in said plane. Where the projection of the head is circular, or substantially circular, in said plane, the pressure applied when the device comes under compression from force applied from the nucleus will apply evenly on the area surrounding the defect. For instance, the head may be hemispherical, elongate, curved, and so on. As such, the device may resemble a mushroom, a T-bar, or an anchor.

The total width of the head (w) is typically tailored to the specific size of the defect to be treated, and as such is not particularly limited. The total width of the head (w) may be from approximately 0.1 to approximately 30 mm, preferably from approximately 0.5 to approximately 20 mm, such as from approximately 1 to approximately 15 mm. Exemplary approximate total widths of the head (w) include: 0.3, 0.5, 0.7, 1.0, 1.7, 3.4, 5.1, 6.8, 8.0, 10.2, 12.0, 15.0, 16.0, 17.0, and 20.0 mm.

The height of the head (h) is typically tailored to the specific size of the defect to be treated, and as such is not particularly limited. The height of the head (h) may be from approximately 0.1 to approximately 20 mm, preferably from approximately 0.5 to approximately 15 mm, such as from approximately 1 to approximately 10 mm. Exemplary approximate heights of the head (h) include: 0.3, 0.5, 0.7, 1.0, 1.7, 3.4, 5.1, 6.8, 8.0, 10.2, 12.0, 15.0, 16.0, and 17.0 mm.

The width of the body is typically adjusted with respect to the size of the head, and so may also vary depending on the size of the defect to be treated. For example, the width of the body may be from approximately 0.01 to approximately 15 mm, preferably from approximately 0.05 to approximately 5 mm, such as from approximately 0.1 to approximately 3 mm.

The length of the body is typically adjusted with respect to the size of the head, and so may also vary depending on the size of the defect to be treated. For example, the length of the body may be from approximately 0.1 to approximately 30 mm, preferably from approximately 0.5 to approximately 20 mm, such as from approximately 1 to approximately 15 mm.

Typically, the body is attached to the surface of the head, typically substantially in the centre of said surface, such that the head extends beyond the body approximately equally in opposing directions or in all directions.

The device of the present invention is directional, and may resemble a plug. The shape and design of the device has two main benefits: it reduces the chance of expulsion of the device itself through the annular defect, and also reduces or prevents further herniation of nuclear contents. Preferably, the device also has adhesive properties, providing more robust expulsion resistance. Furthermore, the design of the device actually means that it has increased resistance to herniation of nuclear contents with increasing load, unlike all externally applied patches.

The device of the present invention is particularly advantageous, and can be modified to have up to three-factor resistance to expulsion, namely short, medium and long term resistance to expulsion. Thus, the device may comprise a sticky material that imbues the device with adhesive properties, particularly adhesion to collagen. Preferably, the device does comprise a sticky material. The adhesive properties of the device provide short term resistance to expulsion. Additionally, the shape of the device results in a physical pressing force against the surrounds of the defect, providing medium term resistance to expulsion. Finally, the device may be susceptible to or even promote growth of cells into material, or generally extra cellular matrix formation around the implant, and this provides long term resistance to expulsion.

The device of the present invention is therefore particular suited to repair of the annulus fibrosus.

The device of the present invention comprises a head and a body. These may be formed simultaneously and be part of one single continuous article, which is preferred. Alternatively, they may be formed separately as two distinct articles that are later joined together prior to or during insertion. Preferably, the head and a body are joined together prior to insertion if they are formed separately.

The device may have a surface coating or chemical modification that enhances bio-adhesion to improve expulsion resistance properties. This modification may be chemical grafting, physical coating or surface modification (e.g. plasma treatment) and could include, but is not limited to, chemistries such as thiols, dopamine and urethanes.

Where a nucleus pulposus implant is present, the device may also bind chemically to said implant. This further increases the resistance of the device to expulsion from the intervertebral disc, and also the device's ability to resist herniation of nuclear material through the annular defect. The nucleus pulposus implant may be implanted before, concurrently, or after the annulus fibrosus repair.

### Hydrogel

The head comprises a deformable nanocomposite hydrogel. The head may also comprise one or more other materials, such as any material that is capable of swelling under physiological conditions. In one embodiment, the head consists of a deformable nanocomposite hydrogel.

The material of the body is not particularly limited, and the body may be made of any biologically compatible material. Preferably, though, the body comprises a deformable nanocomposite hydrogel. In one embodiment, the body consists of a deformable nanocomposite hydrogel. This may be the same as or different to the deformable nanocomposite comprised in the head.

It is particularly preferred that the body and head are formed from the same nanocomposite hydrogel.

The hydrogel comprises a polymer (herein referred to as a hydrogel polymer) of one or more water soluble monomers (a polymer that is obtained from or obtainable by the polymerisation of one or more water soluble monomers). Two or more different hydrogel polymers may be combined, e.g. blended, in the nanocomposite hydrogel.

The hydrogel may be a nanocomposite clay hydrogel such as those described in WO 2013/027051 A1, the contents of which is incorporated herein in its entirety by reference.

The hydrogel polymer is typically a biocompatible, elastomeric polymer with bioinductive and adhesive properties. For instance, the polymer may be polyvinyl acetate, polyethylene glycol, polyvinylpyrrolidone, or a combination of these polymers, or any other thermoresponsive polymer. Unless otherwise indicated, the hydrogel polymer is hydrated, i.e. it is not dehydrated.

Preferably, the hydrogel polymer is a polymer of one or more water soluble monomers selected from the following group or any combination thereof: acrylic acids, acid anhydrides, acrylates, sulfonic acids, vinyl sulfonates, pyrolidones, N-isopropylacrylamide (NIPAM), N,N-dimethylacrylamide (DMAC), glycerol monomethacrylate, hydroxyl ethyl methacrylate, polyethyleneglycomethacrylate, vinyl pyrrolidone, vinyl acetate, ethylene glycol, and/or styrene sulphonic acid.

More preferably, the hydrogel polymer is a polymer of one or more water soluble monomers, the monomers comprising one or more of NIPAM, DMAC, vinyl acetate, ethylene glycol, and/or N-vinyl pyrrolidone, for example the hydrogel polymer may be a polymer of one or more water soluble monomers selected from the following group or any combination thereof. NIPAM, DMAC, vinyl acetate, ethylene glycol, and/or N-vinyl pyrrolidone.

Of these, the most preferred are NIPAM and DMAC. Indeed, it is particularly preferred that hydrogel polymer is a polymer of one or more water soluble monomers, the monomers comprising at least NIPAM and optionally also DMAC. For example, the hydrogel polymer may be a polymer of NIPAM and optionally also DMAC. NIPAM is a suitable monomer to provide the required elasticity of the resulting hydrogel. However, including small amounts of DMAC in the polymer increases the stickiness of the hydrogel, which may enhance adherence of the device to the collagen of the annulus which surrounds the defect following insertion. Accordingly, it is preferred that the polymer is formed of a first water soluble monomer which provides the required elasticity and structural features, e.g. NIPAM, and optionally a second water soluble monomer which increases the stickiness of the hydrogel, e.g. DMAC.

In a preferred aspect, therefore, the polymer comprises a first and a second water soluble monomer, wherein the first and second water soluble monomers are present in a mass ratio of from 100:1 to 3:1, preferably from 50:1 to 5:1, more preferably from 15:1 to 5:1. The first and second monomers may be chosen from any of the monomers identified above. However, it is particularly preferred that the first monomer is NIPAM, and the second monomer is DMAC. For instance, a particularly preferred hydrogel polymer is a polymer of NIPAM and DMAC, where the ratio of NIPAM to DMAC is from 50:1 to 5:1.

The amount of the polymer in the hydrogel is not particularly limited. For instance, it may be from 0.1 to 50 wt. % with respect to the total mass of the hydrogel. Preferably, the amount of the polymer in the hydrogel is from 1 to 30 wt. %, more preferably from 2 to 20 wt. %, even more preferably from 5 to 15 wt. % with respect to the total mass of the hydrogel. For the avoidance of doubt, these amounts refer to the hydrated hydrogel. If the hydrogel has been dehydrated, then these amounts will be correspondingly higher.

One or more radical initiators may be used to polymerise the one or more monomers to obtain the hydrogel polymer. Preferably, one or more radical initiators are used. The one or more radical initiators are not particularly limited, and the skilled person can readily select suitable initiators without undue burden.

Exemplary radical initiators include the following: azobisisobutyronitrile (AIBN), 2,2′-azobis(2-methylpropionamide) (AIBA) and/or cyanovaleric acid (CVA). Preferably, the one or more radical initiators comprise AIBN, AIBA, and/or CVA. More, preferably the one or more radical initiators comprise AIBN. Thus, it is particularly preferred that AIBN is used as a radical initiator in the polymerisation of the one or more water soluble monomers.

The amount of radical initiator tis not particularly limited. Preferably, the amount of the one or more radical initiators in the hydrogel is from 0.001 to 10 wt. %, more preferably from 0.01 to 5 wt. %, even more preferably from 0.05 to 2 wt. % with respect to the total mass of the hydrogel. For the avoidance of doubt, these amounts refer to the hydrated hydrogel. If the hydrogel has been dehydrated, then these amounts will be correspondingly higher.

The hydrogel may comprise one or more crosslinking agents. Preferably, the hydrogel does comprise one or more crosslinking agents.

There is no particular limitation to the nature of crosslinking agents that may be used. For instance, crosslinking agents may utilise one or more of the following interactions: covalent bonds (in particular peptide or ester bonds), ionic bonds, and electrostatic interactions.

The one or more crosslinking agents may be selected from the following group or any combination thereof: clay particles, aldehydes, ketones, carboxylic acids, amines, amides, esters, alkali metal salts and alkali earth metal salts.

Preferably, the one or more crosslinking agents comprise clay particles. More preferably, the one or more crosslinking agents consist of clay particles.

When present, which is preferred, the clay particles may be dispersed and/or exfoliated within the nanocomposite hydrogel. Preferably, the clay particles are dispersed and exfoliated within the hydrogel.

Preferably, the clay particles comprise particles of one or more of the following clays: montmorillonite, hectorite, saponite, vermiculite, mica, bentonite, and/or a fibrous clay. More preferably, the clay particles comprise particles of hectorite. In one embodiment, the clay particles comprise the hectorite Laponite-XLG (Rockwood Ltd.: [Mg_{5.34}Li_{0.66}Si₈O₂₀(OH)₄]Na_{0.66}).

Thus, it is particularly preferred that the hydrogel of the present invention comprises exfoliated hectorite clay particles, such as exfoliated Laponite-XLG.

When added, which is preferred, the crosslinking agent is added in an appropriate amount to achieve the desired degree of crosslinking.

For instance, the amount of crosslinking agent in the hydrogel may be from 0.01 to 20 wt. % with respect to the total mass of the hydrogel. Preferably, the amount of the crosslinking agent in the hydrogel is from 0.1 to 10 wt. %, more preferably from 0.2 to 5 wt. %, even more preferably from 0.5 to 2 wt. % with respect to the total mass of the hydrogel. For the avoidance of doubt, these amounts refer to the hydrated hydrogel. If the hydrogel has been dehydrated, then these amounts will be correspondingly higher.

When the polymers in the hydrogel are crosslinked by clay particles, the individual polymer chains may be anchored at one or both ends to surfaces of the clay particles. The clay particles may therefore act as crosslinks between polymer chains such that the clay particles and polymers are interconnected.

The nanocomposite hydrogel may further comprise one or more additional agents, for example it may contain one or more therapeutic agents. In one embodiment, the nanocomposite hydrogel may further comprise one or more of the following additional agents or any combination thereof: a therapeutic agent (e.g. a drug, mammalian cells such as stem cells, an antimicrobial, or an antibody), a bacteria, a protein, a pharmaceutical, a peptide, another water soluble polymer, hyaluronic acid, hydrophilic gels, polyethylene glycol, polypropylene glycol, a mixture of polyethylene glycol and polypropylene glycol, cellulose ethers, chitosan, alginates, proteoglycans, natural starches, collagen, gelatine, hydroxyapatite, a thickener, a rheology modifier, a surfactant, a pigment, a dye, and/or a radio opaque material. Of these, therapeutic agents such as drugs, mammalian cells such as stem cells, antimicrobials, and antibodies, or combinations of two or more of these, are particularly preferred.

The amount of additional agents in the hydrogel is preferably from 0.01 to 20 wt. % by total mass of the hydrogel, more preferably from 0.05 to 10 wt. %, even more preferably from 0.1 to 5 wt. %.

When present, the one or more additional agents, for instance therapeutic agents such as drugs, stem cells, antimicrobials, and antibodies, may be used to dope the device in order to increase the efficacy of the device, for example by improving recovery, enhancing treatment of damaged tissue, and/or speeding up growth of cells into material, or generally extra cellular matrix formation around the implant.

When the hydrogel comprises one or more therapeutic agents such as a drug or a stem cell, the present invention provides the following: the therapeutic agent for use in a method of treating a condition in a subject caused or exacerbated by the presence of an annular defect in an annulus fibrosus of an intervertebral disc of the subject, the method comprising closing the annular defect by inserting the device of the invention. Similarly, the present invention also provides: the use of the therapeutic agent for the manufacture of a medicament for use in a method of treating a condition in a subject caused or exacerbated by the presence of an annular defect in an annulus fibrosus of an intervertebral disc of the subject, the method comprising closing the annular defect by inserting the device of the invention.

The hydrogel may further comprise a radio opaque material so as to give the device of the invention permanent radiopacity to enable observation during surgical procedure or afterwards at follow up.

Prior to use, the spinal repair device of the present invention may be subject to one or more post-production modification processes. For instance, a surface coating may be applied to the device in order to enhance bio-adhesion. Such a coating is preferably based on thiol, dopamine, and/or urethane chemistry. Preferably, such a coating is achieved via chemical grafting, physical coating, or surface modification such as plasma treatment. In one aspect of the invention, no additional coating is applied to the device. Indeed, this is often preferred. The invention may therefore be beneficial in that no adhesive is required.

### Device Shape

As mentioned above, the spinal repair device of the present invention has a specific shape and design that contribute to its technical advantages. In particular, the device includes at least: a head comprising a surface and a body having a major axis, wherein the body is typically connected to the surface and configured such that its major axis is approximately perpendicular to the surface, the connection between the body and head defining a plane that is perpendicular to the major axis, the projection of the head on the plane extending beyond the projection of the body on the plane.

Preferably, the head is hemispherical in shape and has a curved surface and a flat surface, the body being connected to the flat surface.

During insertion, the device is inserted head-first into the annular defect. It is therefore preferred that the head is deformable to a size that is from about 60% to about 10% of its original size by volume, more preferably from about 50% to about 15%, even more preferably from about a third (33.3%) to about a fifth (20%). The deformation of the head is important in enabling the device of the invention to transfer pressure applied radially outwards by the nucleus away from the annular defect and towards the surrounds of the annular defect, thus locking the device in place and minimising or preventing nuclear material from escaping from the intervertebral disc via the annular defect. The deformation of the head also allows the facile insertion of the device through the annular defect, and crucially also prevents the worsening of the annular defect during insertion. After insertion has been completed, the head then re-shapes to a size and shape that is either the same as, slightly smaller than, or slightly larger than its original size and shape. This may be achieved by various factors, including natural reversion to the devices original size and shape, and enhanced size/shape increase due to swelling under physiological conditions, for instance due to absorption of water. It is therefore important that the head of the device is larger than the annular defect. This mechanically prevents the device from exiting the intervertebral disc when pressure is applied and similarly prevents herniation of the disc contents when a load is applied to the disc. The skilled person can readily select a device head of suitable size based upon the defect to be treated.

The body may also be deformable. In one aspect, the body is deformable. In another aspect, the body is not deformable.

Preferably, the head is curved and/or elongate. For instance, the head may preferably be in the shape of a hemisphere, a bar, or a rainbow.

Preferably, the body is elongate. For instance, the body may be cylindrical in shape.

The device may further comprise other sections. Alternatively, the device may comprise the head and body sections only, which is preferred.

Exemplary overall shapes of the device include a mushroom, a T-bar, and an upside-down anchor.

When the overall shape of the device is a mushroom, the head of the device is hemispherical, and the body of the device is elongate. The surface the body is connected to is approximately flat, and the outer surface of the head which is proximal to the nucleus pulposus in use is rounded. The mushroom shape is particularly good at distributing pressure applied by the nucleus evenly and consistently across the annulus fibrosus. In a particularly preferred aspect, the device has a mushroom shape with a cylindrical body resembling a stem, and a hemispherical head resembling an end cap, wherein the body is connected to the flat face of the hemispherical body.

When the overall shape of the device is a T-bar, the head of the device is elongate and has a major axis that is approximately perpendicular to the major axis of the body. The body is also elongate.

When the overall shape of the device is an anchor, the head of the device is elongate and curved, and may be considered as resembling a rainbow or the like. The body is elongate. The ends of the head curve down towards the body, thus providing a curved surface that the nucleus pulposus can push against during use.

It is particularly preferred that the device is mushroom-shaped.

The size of the device is not particularly limited, and may be readily tailored to suit the size of the particular annular defect to be treated. To this end, as mentioned above, the size of the head must be larger than the annular defect such that the head prevents nuclear material from escaping from the annular defect during use. Similarly, the size of the head must not be too large with respect to the annular defect, otherwise it will damage/worsen the defect on insertion, even when deformed.

### Production Method

The following production methods relate to the production of pre-formed devices of the invention.

The hydrogel of the device of the present invention may be produced in accordance with a method comprising the following steps:
preparing an aqueous suspension containing (i) one or more crosslinking agents, (ii) one or more water soluble monomers, and (iii) one or more radical initiators capable of free radical disassociation to form a composite hydrogel precursor liquid;
dissociating the one or more radical initiators to provide free radical polymerisation of the one or more monomers to produce an aqueous polymer precursor hydrogel, wherein the polymerisation occurs at a temperature above a gelation temperature of the aqueous polymer precursor hydrogel;
optionally maintaining the polymer precursor hydrogel in a fluid phase above the gelation temperature;
processing the polymer precursor hydrogel into the desired shape prior to and/or during the step of reducing the temperature thereof, and
reducing the temperature of the polymer precursor hydrogel below its gelation temperature to form a hydrogel. The crosslinking agents, monomers and radical initiators are those described above.

In one embodiment, where the cross-linking agent comprises clay particles, the hydrogel may be produced in accordance with a method described in WO 2013/027051 A1, the contents of which is incorporated herein in its entirety by reference. In particular, the hydrogel may be produced via a method comprising the following steps:
preparing an aqueous suspension containing (i) dispersed clay particles, (ii) one or more water soluble monomers, and (iii) one or more radical initiators capable of free radical disassociation to form a composite hydrogel precursor liquid;
dissociating the one or more radical initiators to provide free radical polymerisation of the one or more monomers at a temperature above a gelation temperature of a resulting aqueous polymer-clay precursor hydrogel;
optionally maintaining the polymer-clay precursor hydrogel in a fluid phase above the gelation temperature;
processing the polymer-clay precursor hydrogel into the desired shape; and
reducing the temperature of the polymer-clay precursor hydrogel below its gelation temperature;
wherein the precursor is processed prior to and/or during the step of reducing the temperature thereof, and wherein the clay particles, water soluble monomers and radical initiators are as described above.

As will be readily apparent to the skilled person, when the head and body formed individually a separate parts and then connected to produce the device, any of the above methods may be used to form the head and body. For example, the head and body may both comprise the same hydrogel but be formed separately. Alternatively, the head may comprise a first hydrogel and the body may comprise a second hydrogel.

The following temperatures apply to all of the methods described above.

The gelation temperature of the precursor hydrogel is typically in the range of 30 to 40° C., more typically 31 to 39° C., even more typically 33 to 37° C. For instance, the gelation temperature may be around 35° C., although this will depend on the nature of the monomers and crosslinking agents used, as well as the ratios thereof.

Alternatively, gelation may occur at a temperature that is higher or lower than physiological temperatures. For instance, gelation may occur between 4° and 60° C., such as at around 50° C. Or gelation may occur between 10 to 30° C., such as at around 20° C.

The one or more radical initiators preferably dissociate in the temperature range of 40 to 100° C., more preferably 60 to 90° C., even more preferably 75 to 85° C.

The precursor hydrogel is preferably maintained in a fluid phase at a temperature between 37 and 80° C., more preferably between 4° and 70° C., even more preferably between 5° and 65° C., even more preferably still between 55 and 60° C.

The precursor is preferably processed at a temperature between 41 and 80° C., more preferably between 5° and 70° C., even more preferably between 55 and 60° C. Indeed, the precursor may be processed at the same temperature that it is maintained at. This can simplify the production method.

In the final step of the method, the temperature of the precursor is preferably reduced to a temperature in the range of 4 to less than 37° C., preferably 10 to 30° C., preferably 15 to 25° C. For instance, the precursor could be cooled to room temperature in order to set the gel. Whilst even lower temperatures could also be used to set the gel, it is believed that a more gradual cooling step may be beneficial to reduce residual stress in the material.

The reagents/materials for use in the production methods of the invention correspond to those identified above. The amounts of said reagents are not particularly limited, and suitable amounts may be readily determined by the skilled person. Preferred amounts are as indicated above.

The precursor hydrogel may be processed (i.e. shaped to form the above-described shape) by any appropriate method known in the art. For instance, the precursor may be processed by cast moulding, spinning, foaming, coating, 3D printing, or spraying. Preferably, the precursor is processed by cast moulding.

After production, the device of the invention may be used immediately. Alternatively, it may be subject to one or more further modifications, as outlined above. In addition to, or instead of, these modifications, the device may be stored until such time that it is required.

The device of the invention is stable and may be stored under any suitable conditions, which are readily apparent to the skilled person. It is preferred that the device be stored in saline solution at a temperature between 2° and 37° C.

### Annular Defect Repair

The device of the present invention is intended primarily for the repair of annular defects. More specifically, it is used to treat annular defects so as to prevent, or reduce, herniation of nuclear material therefrom. In particular, as described above, the device deforms under pressure from the nucleus pulposus and transfers that pressure to the surrounds of the annular defect, thus preventing the herniation of nuclear material and also locking itself in place. As such, the device of the present invention can be used to help restore proper intervertebral disc function and alleviate a patient's symptoms.

The application of the device of the invention is not particularly limited. For instance, the device can be applied to the closure of annular defects caused by procedures to treat herniation of the nucleus pulposus, such as microdiscectomy. It can also be applied to annular defects that arise de novo and cause symptoms such as back pain and leg pain. It can also be applied as part of a whole disc restoration procedure (when a painful degenerate disc is treated with an implant to the nucleus pulposus through a portal in the annulus fibrosus and the annulus portal needs to be sealed at the end of the procedure).

Most commonly, the device is pre-formed prior to insertion in accordance with the method described above. During implantation, the head of the device is inserted through the annular defect. The head has an elasticity such that it can deform to fit through the annulus defect, and will re-shape following insertion. The head is deformed prior to and/or during insertion, and may also be dehydrated. This reduces the size of the head so that it can pass through the defect without worsening the defect. Once inside the annulus, the head then opens and/or hydrates to a size that is larger than the annular defect. This mechanically prevents the device from exiting the intervertebral disc when pressure is applied and similarly prevents herniation of the disc contents when a load is applied to the disc. Advantageously, the design of the device means that it becomes more likely to resist herniation of nuclear contents with increasing load unlike all externally applied patches.

The device is typically pre-formed and then inserted into the annular defect, e.g. by pushing the device through the defect, head-first, until the head has completely passed through the defect and resides within the nucleus. Typically, the device is formed as a single component comprising the head and the body.

In one aspect, the device may be dehydrated prior to insertion into the annular defect. In addition to, or in place of, deformation, the dehydration reduces the size of the device, thus reducing the likelihood of damaging/worsening the annular defect on insertion. The material of the device is such that it will rehydrate under physiological conditions. After insertion, therefore, the dehydrated device swells, for example it may swell to its original size, or a size that is slightly greater than or smaller than its original size, thus blocking the annular defect and locking the device in place.

The invention therefore includes a spinal repair device obtainable by dehydrating the device described above. In other words, the invention includes a spinal repair device comprising a dehydrated hydrogel, wherein the dehydrated hydrogel is the hydrogel as described above that has subsequently been dehydrated. The invention also includes a method of dehydrating a spinal repair device. This can be achieved by any routine dehydration means known in the art, for instance heating at a temperature of between 30 to 80° C. for around 2 hours to 2 days.

Dehydration may be used as an alternative to deforming the head on insertion. However, it may also be used in addition to deforming the head. Hence, the dehydrated head of the device may also be itself deformed on insertion, thus further reducing the size of the head. This may be particularly useful for annular defects of irregular size and/or shape, wherein a relatively large device head is required to close the defect, but this runs the risk of worsening the defect on insertion.

The invention therefore includes a method of treating a subject suffering from a condition caused or exacerbated by the presence of an annular defect in an annulus fibrosus of an intervertebral disc of the subject. The method comprises closing the annular defect by:
inserting the head of the device of the invention through the annular defect; and
once it is inside the annulus fibrosus, allowing the head to expand to a size greater than the annular defect.

The invention also includes a spinal repair device for use in the treatment of a condition in a subject caused or exacerbated by the presence of an annular defect in an annulus fibrosus of an intervertebral disc of the subject, the method comprising closing the annular defect by:
inserting the head of the device of the invention through the annular defect; and
once it is inside the annulus fibrosus, allowing the head to expand to a size greater than the annular defect.

Optionally, the method also includes an initial step of forming the hydrogel of the invention, wherein the size and shape of the head are selected such that the area of the head is larger than that of the annular defect to be closed. This means that once inside the annulus fibrosus and expanded, the head prevents herniation of the contents of the intervertebral disc through the annular defect.

As mentioned above, administration of the device is by insertion through the defect. If the target site is difficult to access, administration may also be achieved via a cannula that goes to the edge of the defect.

The invention therefore provides a method of repairing an annular defect in a subject, the method comprising inserting the device of the invention into the annular defect such that the body of the device is situated partially or completely in the annular defect and the head is situated inside the annulus fibrosus.

A particular advantage of the method of treatment of the present invention is that it may be less invasive as compared to other known methods, and does not require the use of sutures, surgical screws, or other known fixation means.

As mentioned above, the insertion comprises reducing the size of the head of the device and inserting the head through the annular defect, wherein the size of the head is reduced prior to and/or during the insertion.

Preferably, the size of the head is reduced to from about a third to about a fifth of its original size by volume. The reduction in size of the head means that the insertion does not worsen the defect. The size of the head may be reduced by deforming the nanocomposite hydrogel, and/or dehydrating the nanocomposite hydrogel prior to insertion.

After insertion, i.e. once the head has passed through the defect and is inside the annulus, the head reverts to its original size and shape, or to a size and shape close thereto (which may be slightly smaller or slightly larger). This occurs due to re-opening of the deformed head, and/or also due to swelling which may be caused by a number of factors, most commonly absorption of water. The amount of water absorption will be greater if the device was dehydrated prior to insertion.

The treatment may be part of a whole disc restoration procedure that also involves treating a degenerate disc with an implant to the nucleus pulposus. In such a procedure, the implant may be implanted either before, concurrently or after the device is inserted into the annular defect.

Optionally, the device binds chemically to the implant.

The device can be used to treat any condition caused or exacerbated by annular defects. Exemplary conditions include back pain—in particular lower back pain, neck pain, leg pain, reduced spine function, reduced mobility.

The subject to be treated is preferably a human, but may alternatively be an animal, typically a mammal.

The annular defect may have been caused by a surgical procedure, such as a whole disc restoration or a procedure to treat herniation of the nucleus pulposus, or it may have arisen de novo.

### EXAMPLES

### Example 1: Production of a Spinal Repair Device of the Invention

### 1% Laponite Suspension Synthesis

Laponite-XLG (Rockwood Ltd.: [Mg_{5.34}Li_{0.66}Si₈O₂₀(OH)₄]Na_{0.66}) was used. 10 grams of Laponite-XLG was added to 10 litres of distilled water. The resulting suspension was stirred for a minimum of 24 hours.

### Polymer Synthesis

1% Laponite suspension (40 mL), NIPAM (3.12 g), DMAC (0.48 g) and AIBN (0.04 g) were combined and stirred for a minimum of two hours within a sealed vessel. The mixture was then filtered using a 0.2 m pore syringe filter. The mixture was filtered into a glass vial. The glass vial was sealed and placed within an oven set to 80° C. The mixture was left within the oven for a minimum of 3 hours to ensure complete polymerisation. The resultant polymer was in the form of a colloidal suspension. The temperature of the oven was lowered to 58° C. to ensure that the polymer remained in colloidal form before usage.

### Mushroom Plug Shaped Gels Synthesis

To produce a mushroom-shaped device, a steel mould containing mushroom-shaped outlines was used. The mould was tightly sealed then placed in the oven with the polymer colloid at 58° C. The mould was left in the oven for at least 1 hour. When ready, the mould and polymer colloid were removed from the oven. Each mushroom shaped outline within the mould was filled with polymer colloid. The polymer was allowed to gel over the next 24 hours. After 24 hours, the mould was unlocked and the finished devices released.

### Example 2: Overall Formulation of a Device Prepared in Accordance with Example 1

| Chemical | wt. % |
|---|---|
| Distilled Water | 90.8 |
| Laponite-XLG | 0.901 |
| N-Isopropylacrylamide (NIPAM) | 7.08 |
| Dimethylacrylamide (DMAc) | 1.09 |
| Azobisisobutyronitrile (AIBN) | 0.09 |

1. A spinal repair device including at least:
(a) a head having a surface, and
(b) a body having a major axis;
wherein the body is connected to the surface and the head comprises a deformable nanocomposite hydrogel, the hydrogel comprising polymers of one or more water soluble monomers;
and wherein:
(A) the spinal repair device is for repair of a defect in an annulus fibrosus of an intervertebral disc, the intervertebral disc having an annulus fibrosus and a nucleus pulposus, wherein the body is arranged to be positioned within the defect, and the head is arranged to be positioned within the nucleus pulposus such that its surface is adjacent to the surface of the annulus fibrosus and such that the head extends beyond the periphery of the defect; or
(B) the body is connected to the surface and configured such that its major axis is approximately perpendicular to the surface, the major axis defining a plane perpendicular thereto, the projection of the head on the plane extending beyond the projection of the body on the plane.

2. (canceled)

3. The spinal repair device of claim 1, wherein the body comprises a deformable nanocomposite hydrogel, the hydrogel comprising polymers of one or more water soluble monomers.

4. The spinal repair device of claim 1, wherein the hydrogel further comprises one or more crosslinking agents.

5. The spinal repair device of claim 4, wherein the one or more crosslinking agents are selected from the following group or any combination thereof: clay particles, aldehydes, ketones, carboxylic acids, amines, amides, esters, alkali metal salts and alkali earth metal salts, preferably wherein the one or more crosslinking agents comprise clay particles; or wherein the one or more crosslinking agents are selected from particles of one or more of the following clays: montmorillonite, hectorite, saponite, vermiculite, mica, bentonite, and a fibrous clay, preferably wherein the clay particles comprise particles of hectorite.

6. (canceled)

7. The spinal repair device of claim 1, wherein the hydrogel polymers are polymers of one or more water soluble monomers selected from the following group: acrylic acids, acid anhydrides, acrylates, sulfonic acids, vinyl sulfonates, pyrrolidones, N-isopropylacrylamide (NIPAM), N,N-dimethylacrylamide (DMAC), glycerol monomethacrylate, hydroxyl ethyl methacrylate, polyethyleneglycomethacrylate, vinyl pyrrolidone, vinyl acetate, ethylene glycol, and styrene sulphonic acid, preferably wherein the one or more water soluble monomers comprise NIPAM, DMAC, vinyl acetate, ethylene glycol, and/or N-vinyl pyrrolidone, more preferably wherein the one or more water soluble monomers comprise NIPAM and optionally also DMAC.

8. The spinal repair device of claim 1, wherein the polymer comprises a first and a second water soluble monomer, wherein the first and second water soluble monomers are present in a mass ratio of from 100:1 to 3:1, preferably from 50:1 to 5:1, and wherein the first monomer is preferably NIPAM, and the second monomer is preferably DMAC.

9. The spinal repair device of claim 1, wherein the deformable nanocomposite hydrogel further comprises one or more of the following additional agents: mammalian cells such as stem cells, an antimicrobial, an antibody, a bacteria, a protein, a pharmaceutical, a peptide, another water soluble polymer, hyaluronic acid, hydrophilic gels, polyethylene glycol, polypropylene glycol, a mixture of polyethylene glycol and polypropylene glycol, cellulose ethers, chitosan, alginates, proteoglycans, natural starches, collagen, gelatine, hydroxyapatite, a thickener, a rheology modifier, a surfactant, a pigment, a dye, and a radio opaque material.

10. The spinal repair device of claim 1, wherein the head is hemispherical in shape and has a curved surface and a flat surface, the body being connected to the flat surface.

11. The spinal repair device of claim 1, wherein the head is deformable to a size that is from about a third to about a fifth of its original size by volume.

12. The spinal repair device of claim 1, wherein the device comprises a dehydrated hydrogel, or wherein the device comprises a dehydrated hydrogel which has a total mass that is equivalent to 90 wt. % or less of the hydrated hydrogel.

13-14. (canceled)

15. A method of producing a spinal repair device according to claim 1 comprising the following steps:
preparing an aqueous suspension containing (i) dispersed clay particles, (ii) one or more water soluble monomers, and (iii) one or more radical initiators capable of free radical disassociation to form a composite hydrogel precursor liquid;
dissociating the one or more radical initiators to provide free radical polymerization of the one or more monomers at a temperature above a gelation temperature of a resulting aqueous polymer-clay precursor hydrogel;
optionally maintaining the polymer-clay precursor hydrogel in a fluid phase above the gelation temperature;
processing the polymer-clay precursor hydrogel into the desired shape; and
reducing the temperature of the polymer-clay precursor hydrogel below its gelation temperature;
wherein the precursor is processed prior to and/or during the step of reducing the temperature thereof.

16. (canceled)

17. The method of claim 15, wherein the polymer-clay precursor hydrogel is processed by cast molding, spinning, foaming, coating, 3D printing, or spraying, preferably by cast molding.

18. The method of claim 15, further comprising a dehydration step, optionally wherein the dehydration step takes place after the temperature of the polymer-clay precursor hydrogel has been reduced to below its gelation temperature.

19. A method of repairing an annular defect in a subject, the method comprising inserting the device of claim 15 into the annular defect such that the body of the device is situated partially or completely in the annular defect and the head is situated inside the annulus fibrosus.

20. The method of claim 19, wherein the insertion comprises
reducing the size of the head of the device; and
inserting the head through the annular defect;
wherein the size of the head is reduced prior to and/or during the insertion.

21. The method of claim 20, wherein the size of the head is reduced such that it can be inserted through the defect, preferably wherein the size of the head is reduced to from about a third to about a fifth of its original size.

22. (canceled)

23. The method of claim 20, wherein the size of the head is reduced by;
(A) deforming the nanocomposite hydrogel; or
(B) dehydrating the nanocomposite hydrogel prior to insertion.

24. (canceled)

25. The method of claim 19, wherein the treatment is part of a whole disc restoration procedure that also involves treating a degenerate disc with an implant to the nucleus pulposus.

26. (canceled)

27. The method of claim 25, wherein the device binds chemically to the implant.

28. The method of claim 19, wherein the condition to be treated is one or more of the following: back pain in particular lower back pain, neck pain, leg pain, reduced spine function, reduced mobility.
